(19) United States
(12) Patent Application Publication
(10) Pub. No.: US 2008/0070290 A1
(43) Pub. Date: Mar. 20, 2008

(54) Process for Removing Ammonia, Odours and Dust from Ventilation Air and Apparatus for Use in Such a Process

(75) Inventors: Lise Bonne Guldberg, Roslev (DK); Karl Egeriis Krogshede, Skive (DK)

Correspondence Address: ROBERTS, MLOTKOWSKI & HOBBES, P. O. BOX 10064, MCLEAN, VA, 22102-8064, US

(73) Assignee: SKOV A/S, Roslev (DK)

(21) Appl. No.: 11/909,539

(22) PCT Filed: Mar. 20, 2006
(86) PCT No.: PCT/DK06/00156

§ 371 Date: Nov. 14, 2007

(30) Foreign Application Priority Data
Mar. 23, 2005 (DK) PA 2005 00427

(51) Int. Cl. B01D 53/84; C12M 1/12

(52) U.S. Cl. 435/266; 435/297.2

(57) ABSTRACT

The present invention provides a process and an apparatus which will greatly reduce the amount of emitted ammonia and odour from in particular animal stables by a process which is both efficient, reliable and relatively inexpensive to carry out, and which at the same time removes ammonia to a concentration of 0-2 ppm and the odours to a level of insignificant inconvenience in the ventilation air of a typical pig or poultry stable.

### BACKGROUND OF THE INVENTION

1. Field of the Invention

The present invention provides a process for removing ammonia, odours and dust from ventilation air, in particular from farm buildings. Furthermore the present invention relates to an apparatus for use in such a process.

2. Description of Related Art

Animal production is identified as one of the biggest odour nuisances to local residents and a huge contributor to the emission of ammonia and thereby adding to the increased nutrient loads to the environment. As farming, and in particular animal production, becomes more and more industrialised, the farms have a tendency to grow in size, i.e. more and more animals are kept per unit such that the impact on the adjacent environment is greatly increased. At the same time, there is a general desire in society to provide green areas for leisure purposes, and also the growth of the inhabited areas surrounding the cities are ever increasing, such that the cities continuously moves closer to ever larger farms. It is well-known that farming, and in particular animal production, creates undesirable smells/odours. These odours mainly arise from the manure produced by the animals, and in particular, but not exclusively, ammonia and methane contributes to very offensive smells. Also paracresol (4-methyl-phenol), DMS (mono-, di- and trimethylsulfide), trimethylamine as well as volatile fatty acids, especially propanoic acid, 2-methylpropanoic acid, n-butyric acid, n-valeric acid and iso-valeric acid appears to be main contributors to offensive odours, in particular from pig farms.

Especially in the heavily populated and industrialised countries, the requirements to pollution control are ever increasing. Furthermore, also requirements to the intensity of the smell/odour which a farm may emit to the surrounding environment, is also being regulated with ever increasing requirements for a lowering of these thresholds.

There is, therefore, a need to provide a process and means for reducing the air deriving from the animal stables such that the farm production has a smaller impact on the immediate, adjacent environment.

In stables, and especially in industrialised large-scale production, it is necessary to ventilate off large volumes of air in order to provide the animals, i.e. pigs or poultry, with a suitable environment, where the temperature as well as the availability of fresh air is ensured. Although, as mentioned above, ammonia may be among the most offensive odours deriving from a stable environment, the ventilation air comprises a number of other elements, which together or alone may cause offensive smells.

It is not possible to measure smell, but different methods have been developed in order to determine different smell levels. One such method is the widely used Olfactometer method. Analysis of ventilation air from stables has indicated that there are more than 200 different odour molecules present in the ventilation air in varying concentration, which together gives rise to the highly characteristic smell of a farm. In the western part of the world, the normal requirements to allowable thresholds for smell are therefore not aimed at one or more of these more than 200 different odour generating substances, but are directed to the composite ventilation air, which means that it is the ventilation air as such and not the single components which the thresholds are aimed at.

The present European standard method for determining thresholds utilises a so-called Olfactometer. In an Olfactometer, a number of test persons are exposed to more or less diluted air samples, where the air samples from the ventilation air from a stable have been diluted by clean air. The test persons are selected such that no one has very bad smelling senses, and no one has very good smelling senses, but such that the persons selected are approximately average. As the air sample is diluted less and less with clean air, more and more of the test persons will be able to register that there is some kind of smell in the air stream presented. When half of the test persons are able to smell the odour deriving from the air sample from the stable ventilation air, the threshold for smell has been reached. The threshold, i.e. the odour concentration, is then registered as odour units per cubic meter, which means that an odour concentration of for example 1000 odour units per m³ corresponds to the fact that 1 m³ of ventilation air shall be diluted such that it will fill a volume of 1000 m³ in order to reach the threshold.

Typical values for the odour concentration inside a pig stable vary, but usually they are between a few hundred to a couple of thousand odour units per m³, depending on the circumstances of the stable in question. Although the odour concentration is detectable, it does not indicate anything about the intensity of the odour which also is an important factor for determining how much a particular stable smells. There is a logarithmic relationship between the odour concentration and the intensity, such that it is possible to rate the intensity depending on the odour concentration, and thereby determine limits for, for example, how close pig stables may be placed to city limits or other habitable areas.

hese limits are all decided according to national legislation, but it is obvious that there is a desire to reduce the emitted odours from a stable such that more freedom may be given to the placing of stables and/or placing of housing estates.

In some contries, the legislation is focusing on the single components in the exhaust ventilation air in addition to or instead of an odour concentration determined by the olfactometer method. In these cases, legislation typically contains a list of key malodours, typically some of the key compounds listed above, and a maximum critical concentration belonging to each of the compounds. The concentration of one or more of these listed malodours in the exhaust ventilation air then have to be below the respectively maximum critical concentration.

### OBJECTIVE OF THE INVENTION

It is, consequently, an objective of the present invention to provide a process which will greatly reduce the amount of emitted ammonia, odour and dust from in particular animal stables by a process which is both efficient, reliable, enduring and relatively inexpensive to carry out, and which at the same time reduces up to approximately 95% of the ammonia, odours and dust in the ventilation air of a typical stable. At these highly reduced levels, substantially all nuisance otherwise caused by the ventilation air from animal stables is eliminated.

Another objective of the present invention is to provide an apparatus suitable for carrying out the above air cleaning process.

### BRIEF DESCRIPTION OF THE INVENTION

These objects are addressed in a first aspect of the present invention by providing a process for cleaning air, comprising:
i) contacting a flow of air to be cleaned with a porous carrier medium;
ii) rinsing the carrier medium with an aqueous liquid;
iii) collecting the aqueous liquid used in ii) in a reservoir; wherein
the carrier medium is a material which on its surface comprises colonies of nitrifying bacteria as well as heterotrophic microorganisms; and wherein
the aqueous liquid applied in ii) has a composition which in terms of nutrients ensures the viability and activity of the bacteria and the microorganisms; and wherein
the balance between the different types of bacteria and microorganisms is controlled by performing measurement on the liquid being collected in iii) and, if necessary, wherein the composition of the liquid employed in ii) in terms of dissolved chemical compounds is adjusted on the basis of said measurement.

In a second aspect of the present invention there is provided a process for cleaning air, comprising:
ia) contacting a flow of air to be cleaned with a porous carrier medium of a first filter;
ib) contacting said air with a porous carrier medium of a second filter;
ii) rinsing said carrier medium of each filter respectively with an aqueous liquid;
iii) collecting the aqueous liquid used in ii) in respect of each filter; wherein
the carrier medium of each filter comprises a material which on its surface comprises colonies of autotrophic nitrifying bacteria as well as organoheterotrophic microorganisms; and wherein
the aqueous liquid applied in ii) has a composition which in terms of nutrients ensures the viability and activity of the bacteria and the microorganisms; and wherein
the balance between the different types of bacteria and microorganisms is controlled by performing measurements on the liquid being collected in iii) and, if necessary, wherein the composition of the liquid employed in ii) in terms of dissolved chemical compounds is adjusted on the basis of said measurement.

In a third aspect of the present invention there is provided an apparatus for cleaning air, comprising:
a carrier medium of a porous material which has surface characteristics which enable the colonization, establishment and viability of colonies of nitrifying bacteria as well as heterotrophic microorganisms; said carrier medium being confined in an enclosure; and
means for supplying an air flow into said enclosure so as to contact said air with the surface of said carrier medium; and means for conducting said air, which has been contacted with said carrier medium, out of said enclosure; and
means for rinsing said carrier medium with an aqueous liquid; and
means for collecting said aqueous liquid after rinsing; and
measuring means for measuring characteristics of the liquid which has been used for rinsing; and
controlling means for adjusting the composition of the aqueous liquid which is to be used for rinsing.

In a fourth aspect of the present invention there is provided an apparatus for cleaning air, comprising:
a first filter comprising a carrier medium of a porous material; said porous material having surface characteristics enabling the colonization, establishment and viability of colonies of autotrophic nitrifying bacteria as well as organoheterotrophic microorganisms; and
a second filter comprising a carrier medium of a porous material, said porous material having surface characteristics enabling the colonization, establishment and viability of colonies of autotrophic nitrifying bacteria as well as organoheterotrophic microorganisms; said first filter and said second filter being confined in an enclosure; and
means for supplying an air flow into said enclosure so as to contact said air with the surface of said carrier medium of the first filter; and subsequently so as contact said air with the surface of said carrier medium of the second filter; and
means for conducting said air, which has been contacted with said carrier media of each filter respectively, out of said enclosure; and
means for rinsing said carrier medium of the first filter with an aqueous liquid; and
means for collecting the aqueous liquid obtained after rinsing said carrier medium of the first filter; and
means for rinsing said carrier medium of the second filter with an aqueous liquid; and
means for collecting the aqueous liquid obtained after rinsing said carrier medium of the second filter; and
measuring means for measuring characteristics of the liquid which has been used for rinsing; and
controlling means for adjusting the composition of the aqueous liquid which is to be used for rinsing.

In a fifth aspect of the present invention there is provided a structure comprising an apparatus of the above type.

Finally, a sixth aspect according to the present invention relates to the use of an apparatus of the above type for cleaning of air.

### BRIEF DESCRIPTION OF THE DRAWINGS

FIG. 1 is a schematic representation of an apparatus according to the present invention employing only one carrier medium.

FIG. 2 is a schematic representation of an apparatus according to the present invention employing two carrier media.

FIG. 3 is a detailed representation of an apparatus according to the present invention employing two carrier media.

### DETAILED DESCRIPTION OF THE INVENTION

Ammonia is a dominating component in the air from animal houses e.g. pig houses or poultry houses. Dissolution and oxidation of ammonia affects the ammonia and odour reduction of the filter. When ammonia is oxidised; nitric acid (HNO₃) and nitrous acid (HNO₂) is produced. The acid production is an advantage as acid increases the capacity for the aqueous liquid to bind ammonia and thereby reduce ammonia from the air. When the concentration of acid becomes too high, the activity of the nitrifying bacteria slows down and less acid is produced. This process is known as the “nitrite brake”. Parallel to this process, the produced acid is neutralised when it reacts with ammonia as ammonia dissolution is a base forming process. When the acids are neutralised the inhibiting effect from nitric acid on the ammonia oxidation is reduced and the ammonia oxidising activity increases again. Together, these self-regulating processes ensure that the processes are stabilised.

Although means for cleaning the air is already known, see for example the applicant's prior patent application with publication No. WO 01/93990, which teaches a method for air cleaning in which air is led to the surface of a carrier medium in the form of a biopad comprising bacteria, this system only provides an odour reduction of about 70%.

Tests and further research has however indicated that a reason for this rather low odour reduction is that the conditions provided on the biopad does not result in an optimum balance and activity of the various bacterial species present on said biopad.

It has accordingly been found that in the prior art biopads used for air cleaning one functional group of microorganisms may become the predominant; leading to suppression of other species which may be necessary for an optimum odour reduction, i.e. a proper functioning of the biopad.

Investigations carried out by the inventors have revealed that the surface of the prior art carrier medium—the biopad—comprises a three-layer surface comprising outermost a high density of carbon oxidising bacteria, the second outermost layer comprises high numbers of nitrifying bacteria, and the innermost layer having relatively few active bacteria carrying out anoxic processes.

This stratified distribution suggests that the slow growing autotrophic nitrifyers are continuously overgrown by heterotrophs and that, in case of higher organic loading, the nitrifyers might be deprived of oxygen due to the high heterotrophic activity.

As set forth above it has been found that during use of the prior art biopads, the specific microbial functional groups have occasionally grown in an unbalanced way resulting in the predominance of one species at the expense of other groups, e.g. organoheterotrophic bacteria (carbon oxidising bacteria) versus nitrifying bacteria.

In an efficient air cleaner the organic matter, including the odorants, are decomposed by bacteria and fungi in the outer layer of the biofilm on the carrier medium. In an unbalanced biopad the close contact between microorganisms and ammonia and odourous compounds are reduced. This reduced close contact could occur in case of slime formation and over growth e.g. caused by nutrient limitation. Similar, accumulation of waste products, e.g. nitrite and other metabolites or drying could inhibit the nitrifying and odour reducing microorganisms.

This unbalanced biopad thus reduces its ability to remove odours in an efficient way.

In the investigation carried out by the inventors it was found that the outermost layer, predominantly comprising carbon oxidising bacteria, has a much higher growth rate than the intermediate layer predominantly comprising nitrifying bacteria; thus leading to the suppression of the intermediate bacteria colony.

Furthermore, the outermost carbon oxidising bacteria layer tends to increase in thickness making the access to the air more limited for the intermediate nitrifying bacteria layer.

The result of these facts is that the biopad over time ceases to function properly, and in fact it has been found that especially the nitrogenous compounds passes the biopad without being degraded.

The inventors have now found a way of overcoming the problem of “unbalanced” bacteria colonies in the biopads used for air cleaning.

By the method according to the present invention the biopad is maintained balanced in terms of the desired amount of various bacteria colonies by controlling the composition of the aqueous liquid supplied to said bacteria and microorganisms. With the present control, the composition of the dissolved compounds in the aqueous liquid is kept within ranges that ensures an optimum removal of ammonia and odour of the ventilation.

Thus, by the method of the present invention the state of the bacteria colonies or microorganisms are monitored by performing measurements on the collected liquid which has been used for rinsing the carrier medium, and, if necessary, the composition of the liquid to be supplied to the carrier medium subsequently, in terms of concentration of dissolved compounds, is adjusted on the basis of said measurements.

The Carrier Medium

Generally any kind of porous carrier medium may be applied in the process and apparatus of the present invention. However, the requirement of a relatively free passage of air over the surface of said carrier medium and that said surface is not too smooth must be fulfilled. It is also a requirement that its water binding capacity ensures that the biofilm growing on the carrier medium is kept humid and that waste products can be easily washed away with the rinsing aqueous liquid. Finally it is required that the carrier medium has a high surface area to volume ratio and is very non-biodegradable or optimal inert. The surface area to volume ratio is preferably 300-1000 m²/m³, such as 400-600 m²/m³. Examples of useful carrier media are the bio pads (described below), glass fiber or a material of burned porous expanded clay aggregates.

In a preferred embodiment according to the present invention a biopad is used as carrier medium.

A bio pad may be constructed of corrugated paper of impregnated cellulose in a specific way so as to result in a number of channels being formed. A layer of corrugated paper is at the top and the bottom of the corrugations glued together with a second layer but at another angle. A further layer is glued on, but at the same angle as the first layer. This is continued until an adequate thickness is formed, whereby a block has thus been achieved. The block is then sawn into smaller parts, which results in rectangular blocks. The block is oriented so that the air passes in one direction. The water introduced may run in any direction relatively to the air flow, but a perpendicular direction relative to the air flow is preferred.

In a preferred embodiment of the present invention, the air flows through the porous carrier medium in a substantially horizontal orientation, whereas the liquid used for rinsing the medium flows in a substantially vertical direction.

The filter may also be made from for example burned porous expanded clay granules, such as for example Leca, in that by grading the granules and packing these, it is possible to achieve a filter having a relatively high flow of air through the filter, and an extremely large exposed surface.

As already mentioned above, the air inside a pig stable comprises 100-200 different odorous substances arising from different constituents, but in respect of for example ammonia, which is present in any farm environment, the concentration will typically be 5-25 ppm (parts per million) inside the house. When the ventilation air has passed through the bio pad on which at least the specified two functional types of microorganism are present, i.e. nitrifying bacteria and heterotropic microorganisms, under which operation the bio pad is continually moisturised by water having the desired composition of dissolved compounds, the ammonia concentration measured in the air exiting the bio pad will typically be in the range 0-2 ppm.

A typical cooling-pad is Munter's CELdek 7060, and the supplier's data-sheet indicates that a 100 mm-thick pad exhibits a pressure loss of 12 Pa at a through-going air speed of 1.5 m/s. The pressure loss exponent for the pad is 1.7 (which lies closely to the 1.6 which is given for wood chips (Philips et al. (1995) Journal of Agricultural Engeneering Research, Vol 62: 203-214). The pressure loss can therefore be converted to (0.05/1.5)^{1.7}×12/100 mm=0.37 Pa/m. This is thus 100 times less than earlier bio beds, which in practice means that the process can be effected with very thick bio beds, which may thus have a correspondingly smaller surface. This naturally provides savings in the housing around the bio bed. Alternatively, considerable savings can be achieved in the consumption of electricity for the ventilators.

Due to the construction of the pad, it can be held wet by adding a large amount of water to the top, after which the water distributes itself without dripping at the edges. Hence, the water is distributed as a function of gravity, a high permeability of the filter and the structurally arrangement of the channels in the filter.

The surface to volume ratio of Munters CELdek 7060 cooling pad is 440 m²/m³.

The biopads or any other filters are usually not provided with any of the necessary bacteria and microorganisms when supplied from the manufacturer.

Therefore, it is necessary to establish colonies of such bacteria and microorganisms on the exposed surface of the filter media. This may be done naturally in that the ventilation air comprises the bacteria which may carry out the biochemistry process mentioned with the present invention, or one or more bacteria cultures may be seeded onto the media in order to enhance the performance of the filter in particular during the early periods of operation, but also to promote the growth of particularly preferred bacteria groups. Hence, when used in a pig stable, an efficient amount of bacteria and microorganisme colonies will be formed on the media within 2-6 weeks.

Preferably, the colonies present on the carrier medium are in the form of a biofilm.

It should be noted that in the present application and in the appended claims, the term “contacting a flow of air to be cleaned with a porous carrier medium” is to be interpreted as not necessarily implying that the air is brought into contact with the carrier medium per se. Rather, the expression is meant to imply that the air is brought into contact with the surface of the carrier medium including any biological material present on the surface of the carrier medium, such as a biofilm. Hence, in most situations the above expression is to be interpreted such that “the air to be cleaned is brought into contact with the biofilm present on the carrier medium”

In one preferred embodiment according to the present invention the apparatus and process according to the present invention involves one porous carrier medium. In another preferred embodiment according to the present invention the apparatus and process according to the present invention involves two porous carrier media.

The Microorganisms Present on the Carrier Medium

According to the invention the carrier medium during operation comprises distinct classes of microorganisms. Accordingly, during operation of the process and apparatus according to the present invention, the carrier medium has on its surface colonies of nitrifying bacteria as well as heterotrophic microorganisms. These colonies are preferably present in the form of a biofilm.

In an advantageous embodiment of the invention, the preferred microorganism belongs to the phylogenetic groups Cytophagales, β-Proteobacteria and γ-Proteobacteria and genus Cytophaga, Nitrosomonas and Nitrosospira in addition to a variety of heterotrophic bacteria and fungi.

The thickness of the biofilm is typically 0.15 to 2 mm, but may be up to 5 mm. Most of the ammonia and odour reducing activity is found in the outer zone of the biofilm. This most active zone is typically between 0.02 and 0.35 mm thick and is determined by the penetration depth of oxygen, (O₂). The outer layer and second outermost layer is located within said zone.

The distinct outer surface layer of the biofim contains high numbers of carbon oxidising bacteria. Some of these belong to the phylegenetic groups beta and gamma Proteobacteria. Research indicate that that these heterotrophic bacteria are fast growing bacteria decomposing components from the air; i.e. volatile fatty acids (VFA), amine and similar small components that can easily be taken up. Said components are main odorants in the air.

These heterotropbic bacteria are key elements of the surface of the media as their activity is important in the odour reducing efficiency of the biofilm of the carrier medium of the apparatus of the present invention.

The second outermost layer of the said biofilm contains high numbers of nitrifying bacteria.

In a preferred embodiment of the present invention the second outermost layer of the biopad comprises nitrifying bacteria of the genus Nitrosomonas. The most preferred specie of said layer is Nitrosomonas europea/N. mobilis, which has been found to be the dominant species in the ammonia oxidation; leading to suppression of other species of nitrifying bacteria. It is known, that Nitrosomonas europea are cable to growth with high concentrations of ammonia. However, other nitrifying bacteria of the genus Nitrosospira sp. may also be comprised within the functional group of ammonia oxidizers of said biopads. Nitrosospira are characterised by high substrate affinities (low Kₘ values) in contrast to Nitrosomonas. Thirdly, nitrite oxidising bacteria of the genus Nitrobacter sp may also present in the biofilms.

Nitrifying bacteria in the biofilm are key functional microorganisms of the invention as they are main actors in the ammonia removing function by the processes of ammonia oxidation and possible subsequent nitrite oxidation.

Aditionally, bacteria of the genus Cytophaga may be present in the biofilm. Cytophaga are aerobe chemoorganohetetrophic bactera able to utilize a varity of complex natural polymers e.g. proteins, DNA, RNA, cell walls, lipids, cellulose, agar, chitin, starch, pectin and ceratin. Cytophaga probably take part in reduction of dust and organic matter of microbial origin.

The role of Cytophaga in the biofilm is decomposition of organic matter, especially complex and insoluble matter like dust and organic matter of microbial origin. By doing so the biofilm can decompose the overgrowth and thereby adding to the self-cleaning or “grazing” effect in the biofilm

Research has also demonstrated that fungi may also be present in the said biofilm. Research also indicates that these fungi microorganisms play a role in the odour reducing efficiency in the invention.

The inner layer of said biofilm is anoxic and contains relatively few bacteria.

In addition, a diverse invertebrate fauna, e.g. of insect larvae, nematodes and oligochates found in the biofilm acts as “grazers” on the biofilm. This grazing reduces the thickness of the biofilm in a positive way.

The microorganisms may either be inoculated onto the bio pad, whereby the bio pad may reach its optimal functional capacity much faster in relation to situations where the microorganisms must be present in the stable air, and thereafter colonize and multiply on the bio pad in order to achieve the ammonia and odour cleaning ability as desired.

Measuring Means

In order to ensure the correct balance between the abundances of the colonies of the various bacteria and microorganisms present on the carrier medium, the aqueous liquid used for rinsing the composition of carrier medium is controlled.

In a preferred embodiment according to the process of the present invention, the composition of the rinsing aqueous liquid is controlled by measuring the conductivity of the water used for rinsing.

Preferably the conductivity of the aqueous liquid in the reservoir is maintained at a value of 5-80 milliSiemens/cm, such as 8-60 milliSiemens/cm, for example 10-40 milliSiemens/cm, such as 12-30 milliSiemens/cm, preferably 15-25 milliSiemens/cm, such as 17-23 milliSiemens/cm.

Especially the two genera of micro organism, i.e. Nitrosomonas and Cytophaga have been found to thrive in an environment where the conductivity and thereby the concentration of ammonium, nitrite and nitrate in the reservoir is maintained at a level such that the conductivity preferable will be maintained between e.g. 15 and 23 milliSiemens/cm. The concentration in the reservoir may be adjusted by adding more or less fresh water to the reservoir, and at the same time optionally removing part of the water in the reservoir having a too high concentration of these components, as they influence the liquid conductivity.

The process according to the present invention is extremely reliable in that the biopad as well as the water circulation means are fairly easy to construct, install and maintain, and the control of the proper functioning of the installation is carried out by arranging two electrodes in the water reservoir, and connecting these to a meter for measuring the conductivity of the liquid between the two electrodes. An immediate meter reading will indicated whether or not the system is working optimally. Especially in the conductivity range, 5 to 80 milliSiemens/cm, an optimum compromise has been reached between the amount of water used and the ability to clean the air from ammonia and undesirable odours.

In another preferred embodiment accoding to the present invention the composition of the rinsing aqueous liquid is controlled by measuring the ammonium concentration, ammonia concentration, nitrite concentration, phosphate concentration of the water used for rinsing.

Finally as a secondary measure, the apparatus and the process according to the present invention may also be controlled by using parameters relating to the pressure difference above the filter(s) and and/or one or more of the following parameters: ammonia content and odour degree, e.g., odour units, and specific key odourous compounds e.g. butyric acid, paracresol, mono- di- and trimethyl-phenol and trimethylamine present in the air. That is, in addition to controlling the apparatus be measuring the aqueous liquid used for rinsing, the apparatus may be shut down at regular intervals if measurement of the air entering the apparatus indicates that the quality of the air is above a predetermined threshold limit.

Automated Controlling Means

As set out above, the process and the optimum process conditions are guided with respect to the above parameters of aqueous liquid. It is however in a further advantageous embodiment of the invention possible to additionally monitor other parameters, such as nutrient levels, pH and temperature of the water in the reservoir and pressure above filters, which are controlled within pre-specified intervals, and where the amount or turnover of water being circulated through the porous carrier medium is controlled in relation to the airflow through the medium and the process parameters obtained from the reservoir. By monitoring this, it will be possible to automate the proper operation of the process by providing a computer or other similar means, which will collect the data such as for example the conductivity measured between the two electrodes in the water reservoir, the water flow provided for the carrier medium, and compare these parameters to pre-set intervals, and automatically carry out the necessary adjustments, i.e. removing part of the polluted water in the reservoir and replacing it with fresh water, increasing or decreasing the water flow through the carrier medium, add specific nutrients, etc.

Rinsing with Aqueous Liquid

Such a control unit may also be pre-programmed in order to carry out the cleaning of the porous carrier medium at the pre-specified intervals or circumstances. This cleaning may be carried out by nozzles placed in front of the filters where said nozzles have the ability to create a low pressure jet of water as described below. The controller may also log enough data to provide required documentation for operation.

The amount of water used for rinsing is important in that as the concentration of salts in the reservoir increases, the polluted water containing a rather high concentration of waste product from the ammonia and odour reduction, especially the products ammonium, nitrite and nitrate must be disposed off in some manner. Therefore, it is desirable to produce as small volume of waste water as possible, as the handling of the waste material thereby becomes more economical and easier to dispose off. The polluted water may be used as fertiliser, or may be re-worked in order to be used for other purposes or other types of fertilisers.

Due to the biochemical processes on the carrier medium, the microorganism will substantially turn all ammonia into ammonium, nitrite and nitrate and mineralize organic material into carbon dioxide and inorganic nitrogen and sulphide compounds. Insoluble particles, such as dust, will to a large degree be transported with the water to the reservoir and sediment there.

In addition to having a rinsing water flow which is maintained between certain levels such that a balance is reached between providing sufficient water for the microorganisms to flourish, and at the same time minimising the water usage so that the waste water fraction may be minimised, also the capacity of the carrier medium to let ventilation air pass through is important. Therefore, in a further advantageous embodiment of the invention, the carrier medium comprises a bio pad and the thickness of the bio pad in the flow direction of the air is between 50 and 250 mm, more preferred between 120 and 200 mm, and most preferred approximately 150 mm, and the air speed through the bio pad is less than 1 m/s and preferably approximately 0.8 m/s. With the most preferred thickness, where the bio pad is approximately 150 mm thick, and the ventilation means is arranged such that the air speed through the pad is approximately 0.7-1.0 m per second and most preferred 0,8 m per second, and the air passes two or several filter walls of the bio pad described above and arranged spaced one after the other, the air will be exposed to the microorganisms in each of the bio pad for approximately 0.15-0.19 second per bio pad. This in turn means that the bio pad comprising the two types of microorganisms, where each of the bio pad itself has a thickness of 0.15 m, is capable of passing huge quantities of ventilation air through the bio pad where the ammonia is reduced to less than 2 ppm on average and odour is reduced to a levels which is of no inconvenience for the surrounding neighbours.

In a prefereed embodiment according to the present invention, the aqueous liquid used for rinsing also contains micro- as well as macro-nutrients. Such nutrients may be selected from the group comprising: phosphates, calcium, magnesium, potassium, sodium and/or sulphur ions and vitamins and trace metals, such as Fe—, Cu—, Zn—, Mn—, Co—, I—, Mo— and/or Se-salts.

In case the process for cleaning air is performed with more than one filter, the liquid used for rinsing the first filter may contain such micro- as well as macro-nutrients. Or, as an alternative, the liquid used for rinsing the first filter and also the liquid used for rinsing the second filter may contain such micro- and macronutrients.

In order to maintain optimum working conditions in the carrier medium, particles, and in particular inorganic particles, may be removed at regular intervals in order not to clog up the carrier medium, and thereby reduce the ability of the air to pass through the carrier medium.

Therefore, to avoid occasionally manually procedures where it is necessary to flush the carrier medium in order to clean off agglomerated dust or thick biofilm, an automatic washing robot is preferably placed in front of the filters. The washing robot is composed of one or more nozzles that spray a thick low-pressure jet of water at the filters so that each of the channels which carry the air to be cleaned are flushed by the jet of water. The water from the reservoir is used to flush the filters. The cleaning is carried out at pre-specified intervals and/or as a function of pressure above filters and/or at defined circumstances such as e.g. before a shut down of the air cleaning system in between two batches of pigs in a pig house. The pre-specified intervals of flushing are spaced by 1, 4, 8, 24, 48, 72 or up to 96 hours. The critical limit of pressure above filters that may initiate the washing robot is in the range 20-50 Pa and most preferable 30 Pa.

The cleaning process may be carried out in a rather vigorous manner in that the microorganisms adhere to the carrier medium very strongly, so that a thorough cleaning of the entire carrier medium may be achieved in order to maintain a maximum ventilation capacity, and at the same time the optimum conditions for the microorganisms and thereby the odour and ammonia removal ability of the bio pad may be ensured.

The large amount of water applied to the top means that work can be effected with a water pressure slightly above the lifting height for the water. This means that use can be made of much larger holes in a distributor pipe instead of small holes in the nozzles. It is thus not necessary to have several tanks for the cleaning of the water, a coarse sieve is sufficient. A bio bed is built up of impregnated cellulose and is thus not decomposed by the bacteria. Therefore, it will have a lifetime of 10 years or longer.

On the surface of the pad there is formed a biofilm of microorganisms including ammonia and nitrite oxidising bacteria which converts ammonia to nitrite and maybe subsequently to nitrate. Neither NOx's nor N₂O are formed in significant amounts, as only 0-4,5% and 0-2% of retained N is emitted as N₂O—N or NO—N, respectively.

Tests have shown that the process is not temperature sensitive, in that during the winter period less ventilation is required in order to cool the animals in the stable, such that the bio pad will be exposed to less ventilation air containing the ammnonia and odour molecules, and during the summer when the temperatures are relatively higher, the bacteria maintained in the bio pad will be correspondingly more active, and thereby be able to remove the ammonia and odours from the increased amount of ventilation air through the bio pad.

The invention will now be described in detail with respect to the accompanying drawings, which are only schematic overviews of apparatuses which are suitable for carrying out the process according to the invention.

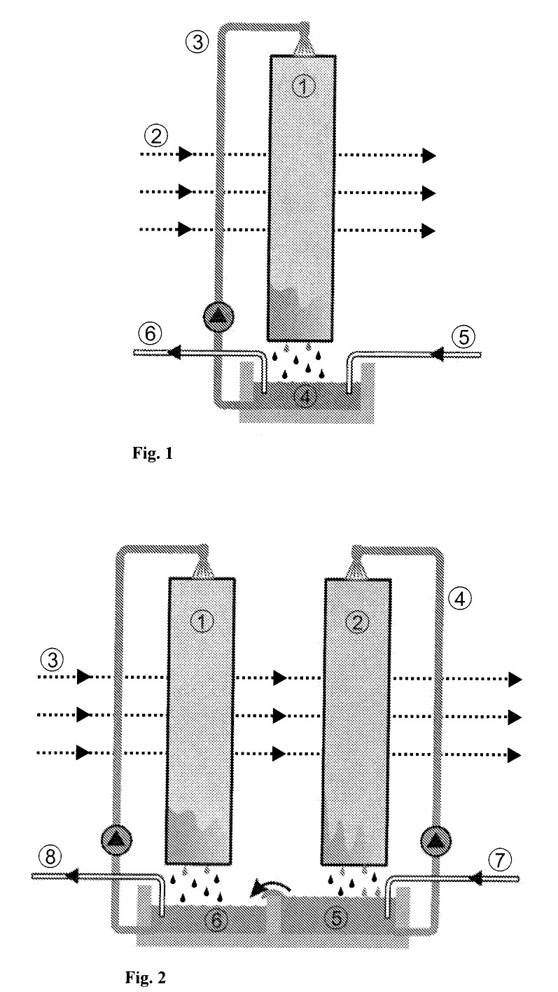

FIG. 1 illustrates the general principle of an apparatus according to the present invention invention, wherein only one filter is employed. A porous carrier media (1) is arranged so that the air (2) to be cleaned will be able to pass through the carrier media. The carrier media is rinsed with an aqueous liquid via pipe (3). The aqueous liquid is collected in a water reservoir (4). It is possible to recirculate the water between carrier media (1) and water reservoir (4). Fresh water (5) is added to the water reservoir. Aqueous liquid is discarded from the system from the water reservoir (4) via pipe (6).

FIG. 2 illustrates the general principle of an apparatus according to the present invention invention, wherein two filters are employed. Two porous carrier media (1) and (2) are arranged so that the air (3) to be cleaned will be able to pass through the carrier media. The two carrier media are spaced. The carrier media are rinsed with an aqueous liquid via pipe (4). The aqueous liquid is collected in a water reservoir (5) and (6). There is one water reservoir for each carrier media. It is possible to recirculate the aqueous liquid to the corresponding carrier media. Fresh water (7) is added to second of the two water reservoirs (5). Aquous liquid to is supplied to the first water reservoir (6) from the second of the two water reservoirs (5). Aqueous liquid is discarded from the system from the first of the two water reservoirs (6) via pipe (8).

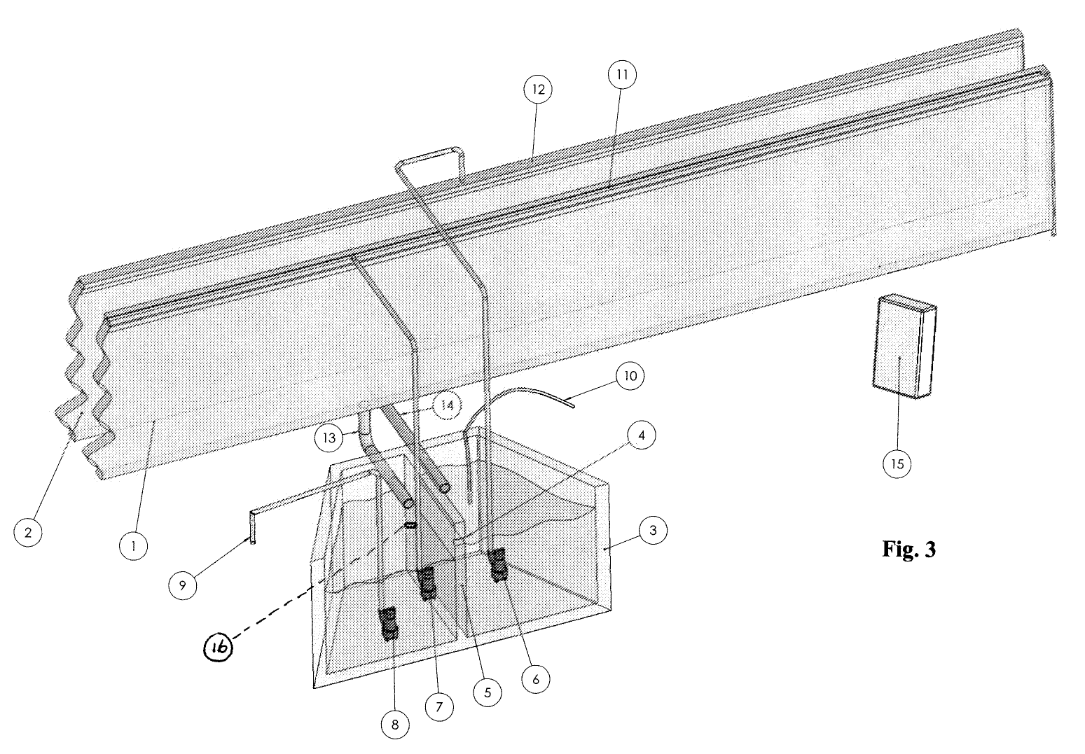

FIG. 3 illustrates the details of a preferred ambodiment of the apparatus of FIG. 2. Two bio pads (1) and (2) are arranged such that the ventilation air will be able to pass through the first bio pad (1) and subsequently through the second bio pad (2). A water reservoir (3) is provided such that via appropriate pumps (6) and (7), piping (11) and (12) it will be possible to recirculate the aqueous liquid from the reservoir and to the top of the bio pads (1) and (2), such that the aqueous liquid, for example by gravity, may moisturise the entire bio pad (1) and (2). The aqueous liquid from the bio pads (1) and (2) is thereafter led through appropriate piping means (13) and (14) back to the water reservoir (3).

The water reservoir (3) is divided in two by a wall (5). Fresh water is supplied to the system through the pipe (10). The aqueous liquid in the part of the tank with the fresh water supply (10) is recirculated from this reservoir through pump (6) and appropriate piping (12) to the second bio pad (2) and is through appropriate piping (14) led back to the water reservoir containing pump (6) and fresh water supply (10).

Through an overflow channel (4), aqueous liquid from the reservoir with fresh water supply is led to the other part of the tank which contains pump (7). The aqueous liquid in the part of the tank containing pump (7) is led from this reservoir through pump (7) and appropriate piping to bio pad (1) and through appropriate piping (13) back to the water reservoir containing pump (7).

A pump (8) pumps aqueous liquid out of the water reservoir and the drained aqueous liquid is discarded through appropriate piping (9).

In order to measure the conductivity of the aqueous liquid supplying bio pad (1), an electrical sensor (16) is provided, which sensor (16) is in electrical contact with a measuring device (15). The measuring device (15) is equipped with a micro processor and interface means, such that in response to the measurements by the sensor (I 6), pump (8) may be running in order to discard strongly polluted aqueous liquid, whereafter another valve on the fresh water line (10) may be opened in order to replenish and thereby dilute the concentration of pollution in the water in reservoir (3).

### EXAMPLE

As an example of one such process and apparatus for removing ammonia, odours and dust from ventilation air, exhaust air from a finisher house was passed through an installation as described above. The installation was built of two spaced rows of carrier media of 10 cm thick Evaporation cooling Pads from Munters. Exhaust air was passed through the filters before it was emitted to the surroundings. The carrier media were dimensioned to an air speed velocity of 0.8 m/sec through filters at max ventilation.

A water reservoir placed next to each filter supplied each filter, respectively, with aqueous liquid. Pumps in each water reservoir in addition to necessary piping and valves ensured recirculation of aqueous liquid from each water reservoir to the respective carrier media. A water distribution pipe line in top of the carrier medias ensured a uniform aqueous liquid distribution along the carrier medias. The amount of aqueous liquid was adjusted in order to keep the carrier medias continuously humid and in the same time to rinse away waste products. Fresh water was added to the water reservoir supplying the carrier media at the back, i.e. the second carrier medium that the exhaust air passed. Waste water was pumped out of the system from the water reservoir supplying water to the carrier media in front, i.e. the first carrier media that the exhaust air passed. The outflow of waste water was controlled in relation to the conductivity measured in the aqueous liquid that recirculated between the carrier media in front and the respective water reservoir. The conductivity sensor was placed in the pipe leading the aqueous liquid from the carrier media in front and back to the respective water reservoir.

When the system was started, the humid conditions on filters and the passing of house air ensured that microorganisms indigenous in exhaust house air colonised the carrier media and established a viable and active biofilm that together with the rinsing of water were able to remove ammonia, odour and dust.

The outflow strategy ensured a minimum outflow. When the conductivity reached a critical limit a given volume of aqueous liquid were pumped out of water reservoir supplying the carrier media in front, i.e. the first carrier medium. The volume of aqueous liquid pumped out of the water reservoir was increased linear as the conductivity increased above the critical limit. After an running-in period the average conductivity was kept in the range of 10-25 mS/cm and typically in the range 17-22 mS/cm.

The efficiency of the process for removing ammonia, odours and dust from the exhaust air and of the apparatus described above for such a process was documented by measuring ammonia, odours and dust in the exhaust air from said finisher house before the exhaust air passed the apparatus and in the exhaust air stream in the outlet, i.e. immediately after said exhaust air had passed the apparatus and before said exhaust air were significantly diluted. The obtained results were as described below.

Ammonia concentration in exhaust air was 30 ppm before it was cleaned and 0 ppm (not detectable, detection limit <0.5 ppm NH₃) after cleaning where said air had passed the two filters. These values were measured regularly during a two months period.

A measure on single components demonstrated the capacity to reduce hydrogen sulphide (CAS 7783-06-04) from 0.0040 ppm to 0.0220 ppm, dimethyl sulphide (CAS 75-18-3) from 0.092 ppm to 0.0190, dimethyl disulphide (CAS 624-92-0) from 0.0650 ppm to <0.0005, methyl mercaptan (CAS 74-93-1) from 0.390 ppm to 0.0065 ppm, trimethylamine (CAS75-50-3) to <0.001 ppm. These values were obtained on a randomly selected day. The amount of volatile fatty acids (VFA) was measured during a period of one month. The VFA propanoic acid (CAS 79-09-4), butanoic acid (CAS 107-92-6), iso-Valeric acid (CAS 503-74-2) and n-Valeric acid (CAS 109-52-4) were all reduced from levels of 10-100 ppm to approx 0.001 ppm.

In another example using a slightly modified apparatus as the one in FIG. 3, average ammonia concentration measured in a two-month period was 9.0 ppm in air before cleaning and 1.2 ppm on average after cleaning. In said installation, but in another two-month period ammonia concentration was 4.3 ppm on average before cleaning and 2.1 ppm on average after cleaning. Odour measurements in the same installation demonstrated odour reduction from 2249 to 1133 OU_{E}/m³, from 1300 to 580 OU_{E}/m³, from 358 to 127 OU_{E}/m³ and from 126 to 105 OU_{E}/m³.

What is claimed is:

1. A process for cleaning air, comprising:
i) contacting a flow of air to be cleaned with a porous carrier medium;
ii) rinsing the carrier medium with an aqueous liquid;
iii) collecting the aqueous liquid used in ii) in a reservoir; wherein
the carrier medium is a material which on its surface comprises colonies of nitrifying bacteria as well as heterotrophic microorganisms; and wherein
the aqueous liquid applied in ii) has a composition which in terms of nutrients ensures the viability and activity of the bacteria and the microorganisms; and wherein
the balance between the different types of bacteria and microorganisms is controlled by performing measurement on the liquid being collected in iii) and, if necessary, wherein the composition of the liquid employed in ii) in terms of dissolved chemical compounds is adjusted on the basis of said measurement characterised in that said measurement relates to the measurement of electric conductivity, and in that the aqueous liquid applied in ii) is adjusted so as to exhibit a conductivity of 5-80 milliSiemens/cm.

2. A process for cleaning air, comprising:
ia) contacting a flow of air to be cleaned with a porous carrier medium of a first filter;
ib) contacting said air with a porous carrier medium of a second filter;
ii) rinsing said carrier medium of each filter respectively with an aqueous liquid;
iii) collecting the aqueous liquid used in ii) in respect of each filter; wherein
the carrier medium of each filter comprises a material which on its surface comprises colonies of autotrophic nitrifying bacteria as well as heterotrophic microorganisms; and wherein
the aqueous liquid applied in ii) has a composition which in terms of nutrients ensures the viability and activity of the bacteria and the microorganisms; and wherein
the balance between the different types of bacteria and microorganisms is controlled by performing measurement on the liquid being collected in iii) and, if necessary, wherein the composition of the liquid employed in ii) in terms of dissolved chemical compounds is adjusted on the basis of said measurement characterised in that said measurement relates to the measurement of conductivity, and in that the aqueous liquid applied in ii) is adjusted so as to exhibit a conductivity of 5-80 milliSiemens/cm.

3. A process according to claim 1, wherein the carrier medium is rinsed continuously.

4. A process according to claim 1, wherein the carrier medium has a ratio surface area/volume of 300-1000 m²/m³, such as 400-600 m²/m³.

5. A process according to claim 1, wherein the carrier medium comprises cellulose, such as paper or paper like material, such as cardboard or paperboard, preferably paper.

6. A process according to claim 5, wherein the carrier medium comprises a pad formed by a plurality of corrugated paper sheets arranged so as to form a plurality of essentially parallel oriented channels through which air can flow.

7. A process according to claim 1, wherein the carrier medium comprises a material of burned porous expanded clay aggregates, or glass fiber sheets.

8. A process according to claim 1, wherein the nitrifying bacteria are selected from the group comprising Nitrosomonas europea, Nitrosomonas mobilis, Nitrosomonas sp, Nitrosospira sp and Nitrobacter sp., preferably Nitrosomonas europea.

9. A process according to claim 1, wherein the heterotrophic microorganism is selected from the group comprising Cytophaga, Beta Proteobacteria, Gamma Proteobacteria.

10. A process according to claim 1, wherein the aqueous liquid to be applied in ii) comprises macro- as well as micro-nutrients.

11. A process according to claim 10, wherein the macro-nutrients comprise phosphates, calcium, magnesium, potassium, sodium and/or sulphur ions.

12. A process according to claim 10, wherein the micro-nutrients comprise vitamins and trace metals, such as Fe—, Cu—, Zn—, Mn—, Co—, I—, Mo-salts and/or Se-salts

13. A process according to claim 2, wherein in step iii) the aqueous liquid originating from rinsing the first filter is collected separately in a first reservoir, and the aqueous liquid originating from rinsing the second filter is collected separately in a second reservoir; and wherein a flow of liquid is established from the second reservoir to the first reservoir; and wherein the second reservoir is replenished with fresh water; and wherein a portion of the aqueous liquid contained in the first reservoir is discarded; and wherein in step ii) the carrier medium of the first filter is rinsed with an aqueous liquid originating from the first reservoir; and wherein the carrier medium of the second filter is rinsed with an aqueous liquid originating from the second reservoir, so as to represent a counter-current rinsing process.

14. A process according to claim 2, wherein in step iii) the aqueous liquid originating from rinsing the first filter is collected separately in a first reservoir, and the aqueous liquid originating from rinsing the second filter is collected separately in a second reservoir; and wherein a flow of liquid is established from the second reservoir to the first reservoir; and wherein a portion of the aqueous liquid contained in the first reservoir is discarded; and wherein in step ii) the carrier medium of the first filter is rinsed with an aqueous liquid originating from the first reservoir; and wherein the carrier medium of the second filter is rinsed with fresh water, so as to represent a counter-current rinsing process.

15. A process according to claim 1, wherein at least part of the aqueous liquid collected in iii) is re-circulated so as to be used in ii).

16. A process according to claim 1, wherein in step iii) the aqueous liquid originating from rinsing the filter is collected in a reservoir; and wherein a portion of the aqueous liquid contained in the reservoir is discarded; and wherein the reservoir is replenished with fresh water; and wherein in step ii) the carrier medium of the filter is rinsed with an aqueous liquid originating from the reservoir.

17. A process according to claim 1, wherein in step iii) the aqueous liquid originating from rinsing the filter is collected in a reservoir; and wherein a portion of the aqueous liquid contained in the reservoir is discarded; and wherein in step ii) the carrier medium of the filter is rinsed with fresh water.

18. A process according to claim 13, wherein the measurement of the aqueous liquid is either performed on the liquid exiting that filter which (in case of more than one filter) is the first filter to be contacted with the air to be cleaned on its way to its corresponding reservoir, or on the liquid present in said corresponding reservoir.

19. A process according to claim 1, wherein the measurement to be performed on the aqueous liquid which is collected in iii) relates to measurement of one or more of the following parameters: ammonia concentration, ammonium concentration, nitrite concentration, phosphate concentration.

20. A process according to claim 1, wherein the aqueous liquid applied in ii) is adjusted so as to exhibit a conductivity of 8-60 milliSiemens/cm, for example 10-40 milliSiemens/cm, such as 12-30 milliSiemens/cm, preferably 15-25 milliSiemens/cm, such as 17-23 milliSiemens/cm.

21. A process according to claim 1, wherein the air to be cleaned comprises ammonia and/or other odour compounds.

22. A process according to claim 1, wherein the air to be cleaned is air originating from a stable, especially a pig or poultry stable or from a manure composting plant.

23. An apparatus for cleaning air, said apparatus being specifically adapted to performing the process according to claim 1; and comprising:
a carrier medium of a porous material which has surface characteristics which enable the colonization, establishment and viability of colonies of nitrifying bacteria as well as heterotrophic microorganisms; said carrier medium being confined in an enclosure; and
means for supplying an air flow into said enclosure so as to contact said air with the surface of said carrier medium; and means for conducting said air, which has been contacted with said carrier medium, out of said enclosure; and
means for rinsing said carrier medium with an aqueous liquid; and
means for collecting said aqueous liquid after rinsing; and
measuring means for measuring characteristics of the liquid which has been used for rinsing; and
controlling means for adjusting the composition of the aqueous liquid which is to be used for rinsing characterised in that said measuring means comprises conductivity controlling means configured to adjusting the composition of the aqueous liquid which is to be used for rinsing in order to maintain a conductivity of said liquid of 5-80 milliSiemens/cm.

24. An apparatus for cleaning air, in particular air from pig stables, said apparatus being specifically adapted to performing the process according to claim 2; and comprising:
a first filter comprising a carrier medium of a porous material; said porous material having surface characteristics enabling the colonization, establishment and viability of colonies of autotrophic nitrifying bacteria as well as organoheterotrophic microorganisms; and
a second filter comprising a carrier medium of a porous material, said porous material having surface characteristics enabling the colonization, establishment and viability of colonies of autotrophic nitrifying bacteria as well as organoheterotrophic microorganisms; said first filter and said second filter being confined in an enclosure; and
means for supplying an air flow into said enclosure so as to contact said air with the surface of said carrier medium of the first filter; and subsequently so as contact said air with the surface of said carrier medium of the second filter; and
means for conducting said air, which has been contacted with said carrier media of each filter respectively, out of said enclosure; and
means for rinsing said carrier medium of the first filter with an aqueous liquid; and
means for collecting the aqueous liquid obtained after rinsing said carrier medium of the first filter; and
means for rinsing said carrier medium of the second filter with an aqueous liquid; and
means for collecting the aqueous liquid obtained after rinsing said carrier medium of the second filter; and
measuring means for measuring characteristics of the liquid which has been used for rinsing; characterised in that said measuring means comprises conductivity controlling means configured to adjusting the composition of the aqueous liquid which is to be used for rinsing in order to maintain a conductivity of said liquid of 5-80 milliSiemens/cm.

25. An apparatus according to claim 23, further comprising means for re-circulating the aqueous liquid used for rinsing so as to be reused for rinsing.

26. An apparatus according to claim 23, wherein the carrier medium has a ratio surface area/volume of 300-1000 m²/m³, such as 400-600 m²/m³.

27. An apparatus according to claim 23, wherein the carrier medium comprises cellulose, such as paper or paper like material such as cardboard or paperboard, preferably paper.

28. An apparatus according to claim 27, wherein the carrier medium comprises a pad formed by a plurality of corrugated paper sheets arranged so as to form a plurality of essentially parallel oriented channels through which air can flow.

29. An apparatus according to claim 23, wherein the measuring means furthermore relates to measurement of one or more of the following parameters: ammonium concentration, ammonia concentration, nitrite concentration, phosphate concentration,

30. An apparatus according to claim 24 comprising a first reservoir for receiving aqueous liquid originating from rinsing the first filter; and a second reservoir for receiving aqueous liquid originating from rinsing the second filter; and means for establishing a flow of liquid from the second reservoir to the first reservoir; and means for replenishing the second reservoir with fresh water; and means for discarding a portion of the aqueous liquid contained in the first reservoir; and means for rinsing the carrier medium of the first filter with an aqueous liquid originating from the first reservoir; and means for rinsing the carrier medium of the second filter with an aqueous liquid originating from the second reservoir, so as to represent a counter-current rinsing process.

31. An apparatus according to claim 24 comprising a first reservoir for receiving aqueous liquid originating from rinsing the first filter; and a second reservoir for receiving aqueous liquid originating from rinsing the second filter; and means for establishing a flow of liquid from the second reservoir to the first reservoir; and means for discarding a portion of the aqueous liquid contained in the first reservoir; and means for rinsing the carrier medium of the first filter with an aqueous liquid originating from the first reservoir; and means for rinsing the carrier medium of the second filter with fresh water, so as to represent a counter-current rinsing process.

32. An apparatus according to claim 23 comprising a reservoir for receiving aqueous liquid originating from rinsing the filter; and means for discarding a portion of the aqueous liquid contained in said reservoir; and means for replenishing the reservoir with fresh water; and means for rinsing the carrier medium of the filter with an aqueous liquid originating from said reservoir.

33. An apparatus according to claim 23 comprising a reservoir for receiving aqueous liquid originating from rinsing the filter; and means for discarding a portion of the aqueous liquid contained in said reservoir; and means for rinsing the carrier medium of the filter with fresh water.

34. An apparatus according to claim 30, comprising means for performing measurements either on the liquid exiting that filter which (in case of more than one filter) is the first filter to be contacted with the air to be cleaned on its way to its corresponding reservoir, or on the liquid present in said corresponding reservoir.

35. An apparatus according to claim 23, furthermore comprising means for control by using parameters relating to the pressure above the filter(s) and and/or one or more of the following parameters: ammonia and odour e.g. odour units, and specific key odourous compounds e.g. butyric acid, paracresol, mono- di- and trimethyl-phenol and trimethylamine

36. A structure comprising an apparatus according to claim 23.

37. Use of an apparatus according to claim 23, for cleaning of air, especially air in a stable, such as a pig or a poultry stable, or a manure composting plant.
